(19) United States
(12) Patent Application Publication
(10) Pub. No.: US 2025/0122516 A1
(43) Pub. Date: Apr. 17, 2025

(54) USE OF GENE ZmNPF6.8 IN REGULATION OF NITROGEN UPTAKE AND UTILIZATION EFFICIENCY OF ZEA MAYS L. AND YIELD PER ZEA MAYS L. PLANT

(71) Applicant: JIANGSU ACADEMY OF AGRICULTURAL SCIENCES, Nanjing (CN)

(72) Inventors: Ling ZHOU, Nanjing (CN); Wei XIONG, Nanjing (CN); Han ZHAO, Nanjing (CN)

(73) Assignee: JIANGSU ACADEMY OF AGRICULTURAL SCIENCES, Nanjing (CN)

(21) Appl. No.: 18/426,394

(22) Filed: Jan. 30, 2024

Related U.S. Application Data
(63) Continuation of application No. PCT/CN2023/125883, filed on Oct. 23, 2023.

(30) Foreign Application Priority Data
Oct. 16, 2023 (CN) 202311335639.0

(51) Int. Cl. C12N 15/82; C07K 14/415

(52) U.S. Cl. CPC C12N 15/8243; C07K 14/415; C12N 15/8216; C12N 15/8261

(57) ABSTRACT

Use of a gene ZmNPF6.8 in regulation of a nitrogen uptake and utilization efficiency of Zea mays L. and a yield per Zea mays L. plant is provided. The gene ZmNPF6.8 has: 1) a coding region nucleotide sequence shown in SEQ ID NO: 3; or 2) a nucleotide sequence that has 90% or more homology with the coding region nucleotide sequence shown in SEQ ID NO: 3 and encodes a same functional protein as the coding region nucleotide sequence shown in SEQ ID NO: 3. In the present disclosure, a Mutator transposon can be inserted into an amino acid-coding region of the gene ZmNPF6.8 to down-regulate an expression level of the gene and significantly reduce a nitrogen uptake and utilization efficiency of Zea mays L. and a yield per Zea mays L. plant.

### CROSS REFERENCE TO THE RELATED APPLICATIONS

This application is a continuation application of International Application No. PCT/CN2023/125883, filed on Oct. 23, 2023, which is based upon and claims priority to Chinese Patent Application No. 202311335639.0, filed on Oct. 16, 2023, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named GBBTZX001-PKG_Sequence_Listing.xml, created on Jan. 12, 2024, and is 16,702 bytes in size.

### TECHNICAL FIELD

The present disclosure relates to the technical field of bioengineering, and specifically relates to a gene ZmNPF6.8 associated with an efficient nitrogen uptake trait of Zea mays L., especially a yield and nitrogen transport of Zea mays L., and use of the gene.

### BACKGROUND

As one of the most important multi-purpose crops globally, Zea mays L. is widely used in food, feed, chemical, biological energy, and other industries. Less than 30% of the nitrogen fertilizer applied in Zea mays L. cultivation is assimilated and utilized, and up to 70% of the nitrogen fertilizer is wasted. The loss of nitrogen fertilizers in large quantities has caused a serious waste of environmental resources.

Nitrogen is a major component of various secondary metabolites such as nucleic acids, chlorophyll, proteins, and plant hormones and plays different roles in the growth, development, and yield formation of Zea mays L. The yield of Zea mays L. is largely determined by the uptake and utilization of nitrogen by Zea mays L. NPF genes, a class of genes for membrane proteins with 12 α-helix transmembrane structures, are widely involved in the uptake and utilization of nitrogen in plants. NPF genes play an important role in improving nitrogen utilization efficiency and yield-associated traits in crops. Therefore, the research on NPF genes has an important breeding application value for improving efficient utilization of nitrogen in Zea mays L. and sustainably improving the yield of Zea mays L.

Functions of many NPF genes in Arabidopsis thaliana (31 NPF genes) and Oryza sativa L. (16 NPF genes) have been clarified, but biological functions of only 4 NPF genes in Zea mays L. have been reported. Compared with the research on Arabidopsis thaliana and Oryza sativa L., biological functions of NPF genes of Zea mays L. in the nitrogen utilization efficiency still need to be explored and clarified. These NPF genes are closely correlated with not only nitrogen uptake in Zea mays L, but also root system building and grain development of Zea mays L. Currently, the exploration of NPF genes in Zea mays L. is not sufficient, and different NPF genes may show different action mechanisms and different contributions to yield variation. Therefore, it is necessary to conduct in-depth research on NPF genes in Zea mays L. that affect nitrogen uptake and transport efficiency and yield.

### SUMMARY

An objective of the present disclosure is to provide use of a gene ZmNPF6.8 in regulation of a nitrogen uptake and utilization efficiency of Zea mays L. and a yield per Zea mays L. plant. In the present disclosure, it has been found through survey analysis for genomic variations of genetic populations and nitrogen content traits of corresponding materials that the gene ZmNPF6.8 of Zea mays L. is related to a nitrogen uptake and transport trait of Zea mays L., and a mutant material with a transposon Mutator inserted is used to verify that a change of an expression level of the gene ZmNPF6.8 can have an impact on a nitrogen utilization rate of Zea mays L. and a yield per Zea mays L. plant.

Specifically, the present disclosure provides the following technical solutions:

The present disclosure provides use of a gene ZmNPF6.8 in regulation of a nitrogen uptake and utilization efficiency of Zea mays L. and a yield per Zea mays L. plant.

The present disclosure also provides use of a gene ZmNPF6.8 in breeding of a maize variety with an efficient nitrogen uptake and a high yield.

The gene ZmNPF6.8 has a nucleotide sequence described in (1) or (2) below:
(1) a coding region nucleotide sequence shown in SEQ ID NO: 3; and
(2) a nucleotide sequence that has 90% or more homology with the coding region nucleotide sequence shown in SEQ ID NO: 3 and encodes a same functional protein as the coding region nucleotide sequence shown in SEQ ID NO: 3.

Or, the gene ZmNPF6.8 has a nucleotide sequence shown in SEQ ID NO: 2, which includes untranslated region (UTR) and intron sequences of the gene ZmNPF6.8.

A protein encoded by the gene ZmNPF6.8 has an amino acid sequence described in (1) or (2) below:
(1) an amino acid sequence shown in SEQ ID NO: 1; and
(2) an amino acid sequence that is obtained through substitution and/or deletion and/or addition of one or more amino acid residues based on the amino acid sequence shown in SEQ ID NO: 1 and has 90% or more homology with and a same function as the amino acid sequence shown in SEQ ID NO: 1.

In the present disclosure, a full-length sequence of the gene ZmNPF6.8 is successfully cloned, as shown in SEQ ID NO: 3, and a corresponding binary vector is constructed and transformed into Nicotiana tabacum L. and Zea mays L. protoplasts. Test results show that the gene ZmNPF6.8 can encode a membrane protein.

In the present disclosure, an expression level of the protein encoded by the gene ZmNPF6.8 is changed to regulate the nitrogen uptake and utilization efficiency of Zea mays L. and the yield per Zea mays L. plant; and the expression level of the protein encoded by the gene ZmNPF6.8 is changed by:
(1) adding a strong promoter element Ubiquitin or CaMV35S;
(2) changing the expression of the gene ZmNPF6.8 through mutagenesis of a transposon, a fast neutron, and ethyl methane sulfonate (EMS); and
(3) changing the expression of the gene ZmNPF6.8 through interference, silencing, inhibition, targeted knockout, or site-directed mutagenesis.

In the present disclosure, a Mutator transposon insertion method is adopted to change expression levels of mRNA with a normal activity of the gene ZMNPF6.8 and the protein encoded by the gene ZMNPF6.8. Since a nitrogen uptake and utilization efficiency and a yield of Zea mays L. can be regulated by changing an expression level of the gene, a Zea mays L. variety and material with a high nitrogen uptake efficiency and a high yield can be cultivated.

The expression level of the gene ZmNPF6.8 in Zea mays L. can be changed through an expression cassette, an enhancer sequence, gene knockout, or the like to affect a nitrogen uptake and utilization efficiency of Zea mays L. and a yield per Zea mays L. plant and cultivate a brand-new maize variety with a high nitrogen uptake efficiency and a high yield.

In addition, an expression cassette, a recombinant vector, a transgenic cell line, or a transgenic recombinant bacterium carrying the gene ZmNPF6.8 in Zea mays L., or molecular biotechnologies, such as a technology for modifying the gene ZmNPF6.8 in Zea mays L. or changing an expression level of the gene ZmNPF6.8 in Zea mays L. through site-directed mutagenesis of a promoter sequence, all fall within the protection scope of the present disclosure.

A plant expression vector includes a binary Agrobacterium tumefaciens vector and a vector that can be used for microprojectile bombardment of plants. The plant expression vector may include a 3′ UTR of an exogenous gene, that is, it includes a polyadenylic acid signal and any other DNA fragment involved in mRNA processing or gene expression. The polyadenylic acid signal can guide the addition of polyadenylic acid to a 3′ terminus of a mRNA precursor, for example, transcribed 3′ UTRs of an Agrobacterium tumefaciens crown gall-induced (Ti) plasmid gene (such as a nopaline synthase gene) and a plant gene (such as a soybean storage protein gene) all have a similar function. When the gene is used to construct a recombinant plant expression vector, any enhanced promoter or constitutive promoter can be added before a transcription initiation nucleotide of the gene, such as a 35S promoter of cauliflower mosaic virus (CAMV) and an ubiquitin promoter of Zea mays L., which can be used alone or in combination with another plant promoter.

In addition, when the gene of the present disclosure is used to construct a plant expression vector, an enhancer including a translation enhancer or a transcription enhancer can also be used, and an enhancer region may be a ATG start codon or an adjacency region start codon, but must be the same as a reading frame of a coding sequence to ensure the correct translation of the entire sequence. The translation control signal and start codon are widely available, and can be either natural or synthetic. A translation initiation region can be derived from a transcription initiation region or a structural gene.

In order to facilitate identification and screening of a transgenic plant cell or a plant, the plant expression vector used can be processed, for example, a gene that can be expressed in a plant and can encode an enzyme allowing a color change or a luminous compound (a GUS gene, a luciferase gene, or the like), an antibiotic marker with resistance (a gentamicin marker, a kanamycin marker, or the like), or a chemical reagent-resistant marker gene (such as a herbicide-resistant gene) can be added. Given the safety of transgenic plants, it is possible to directly screen transformed plants under stress without adding any selectable marker gene.

In the present disclosure, the constructed Zea mays L. ChinaMu transposon-inserted mutant library (Liang et al., 2019) is queried to obtain a Mu transposon insertion position of the gene ZmNPF6.8, and a query result shows that one Mu insertion (zmnpf6.8) occurs in an exon region 4 of the gene ZmNPF6.8. In order to exclude the influence of insertion of a transposon into other sites, a mutant material is backcrossed with B73 for two generations, and a zmnpf6.8 homozygous mutant and a wild-type (WT) material are acquired. Compared with a nitrogen uptake and utilization efficiency and a yield per plant of WT Zea mays L., a nitrogen uptake and utilization efficiency and a yield per plant of a Zea mays L. mutant decrease to varying degrees, indicating that the gene ZmNPF6.8 has an important impact on nitrogen utilization and yield formation of Zea mays L.

Compared with the prior art, the present disclosure has the following beneficial effects:

The present disclosure has found that the gene ZmNPF6.8 of Zea mays L. can regulate a nitrogen uptake and utilization efficiency of Zea mays L. and affect the growth and yield formation of Zea mays L. The down-regulation of an expression level of the gene can reduce a nitrogen uptake and utilization efficiency and a yield per plant of Zea mays L., indicating that the expression level of the gene is positively correlated with the nitrogen uptake and utilization efficiency and the yield per plant of Zea mays L. and is a major influencing factor for the nitrogen uptake and utilization efficiency and the yield per plant of Zea mays L. A protein encoded by the gene and a coding region of the gene can be used for genetic improvement of a plant.

In the present disclosure, a nitrogen uptake and utilization efficiency of Zea mays L. and a yield per Zea mays L. plant can be regulated by changing an expression level of the gene ZmNPF6.8, which is of great significance for future breeding of a Zea mays L. variety with a high nitrogen uptake and utilization efficiency and a high yield.

### BRIEF DESCRIPTION OF THE DRAWINGS

FIGS. 1A-1C show analysis results of expression patterns of the gene ZmNPF6.8;

FIGS. 2A-2B show analysis results of nitrogen uptake and utilization efficiency traits of two ZmNPF6.8 haplotype materials;

FIGS. 3A-3C show a transposon insertion site and fluorescence quantification-based relative expression analysis results of a ZmNPF6.8 gene mutant line;

FIGS. 4A-4E show nitrogen uptake and utilization analysis results of a ZmNPF6.8 mutant line at a seedling stage; and

FIGS. 5A-5C show analysis results of an impact of a ZmNPF6.8 mutation on a yield of Zea mays L.

### DETAILED DESCRIPTION OF THE EMBODIMENTS

The technical solutions of the examples of the present disclosure are clearly and completely described below with reference to the accompanying drawings in the examples of the present disclosure. Apparently, the described examples are merely some rather than all of the examples of the present disclosure. All other examples derived by those of ordinary skill in the art based on the examples of the present disclosure without creative efforts should fall within the protection scope of the present disclosure.

Unless otherwise specified, all experimental methods in the following examples are conventional methods. Unless otherwise specified, the test materials used in the following examples are all commercially available from conventional biochemical reagent stores.

### Example 1 Expression Patterns of a Gene ZmNPF6.8;

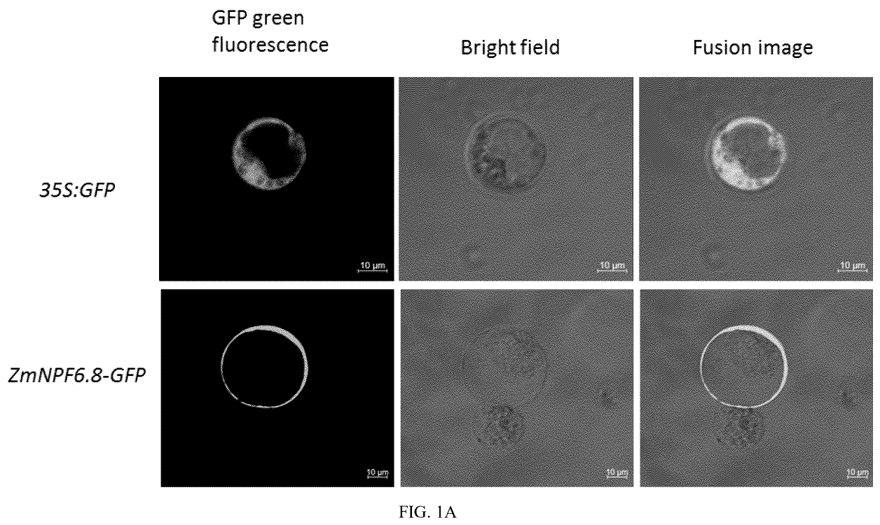

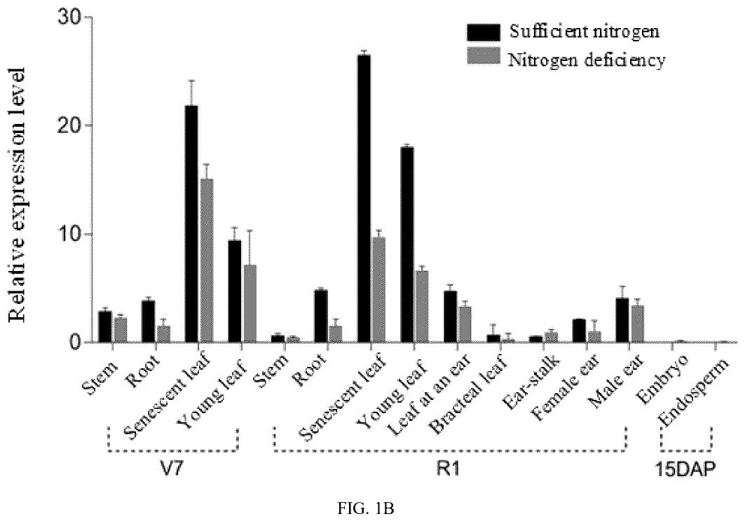

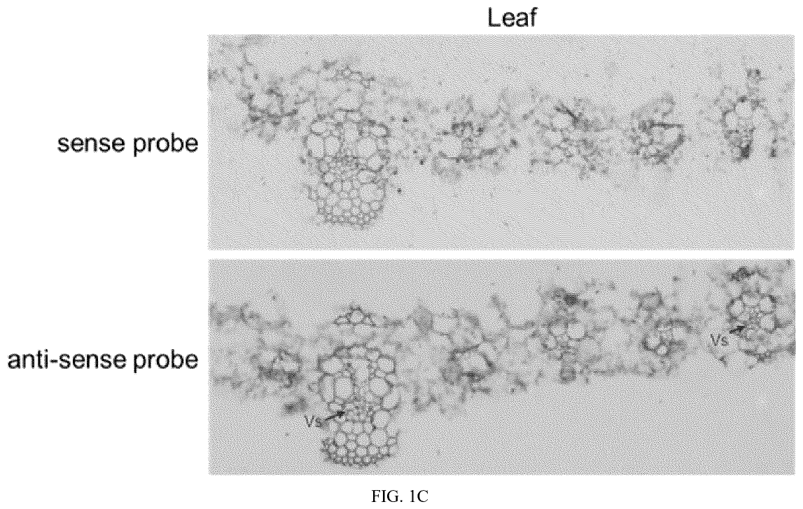

A B73 inbred line was used to conduct an expression pattern study. A ZmNPF6.8-GFP fusion protein was constructed and transiently expressed in a B73 Zea mays L. mesophyll cell protoplast for homology verification of subcellular localization, and a blank vector was adopted as a control group. Experimental results showed that a large amount of green fluorescent protein (GFP) was present inside the protoplast in the control group, while a ZmNPF6.8 (Zm00001d016982)-GFP fluorescence signal was abundantly present on a membrane of the protoplast in the experimental group with the fusion protein, indicating that the ZmNPF6.8 protein is a membrane protein (FIG. 1A).

A CFX Connect Real-Time PCR System (Bio-Rad Laboratories) was then used to conduct qRT-PCR analysis by a two-step method: denaturation: 95° C. for 30 s; and annealing and extension: 60° C. for 30 s, with 40 cycles. A dissolution curve was plotted under the following conditions: 95° C. for 15 s, 60° C. for 60 s, and 95° C. for 15 s, and used for quality control of a fluorescence quantification Cq value. With a housekeeping gene ZmUPF1 (Zm00001d006438) as an internal reference gene, expression levels of the gene ZmNPF6.8 in tissues at different growth stages were calculated according to a 2-ΔΔCt method and subjected to multi-comparison through least significant difference (LSD) of one-way analysis of variance (ANOVA) in the SPSS software (p <0.05). Analysis results showed that the gene ZmNPF6.8 was mainly expressed in Zea mays L. leaves at a high level (SN: 5 mM KNO₃; and DN: 0.5 mM KNO₃) (FIG. 1B).

Finally, RNA in situ hybridization analysis was conducted: B73 seeds were allowed to germinate and then cultivated under hydroponic conditions for 14 d, and leaves and root systems were sampled separately, where three independent biological replicates were set for each sample. Young ears with the highest ZmNPF6.8 gene expression level were fixed in a 50% FAA fixation solution, vacuumed, subjected to gradient ethanol dehydration and gradient xylene permeabilization, embedded in paraffin, and sectioned, and resulting paraffin sections were dewaxed, acetylated, subjected to gradient ethanol dehydration, and then subjected to probe hybridization. A tissue site with a hybridization signal for an antisense probe and without a hybridization signal for a sense probe was an expression site of the target gene in vivo. In situ hybridization results of this study showed that the gene ZmNPF6.8 was expressed at a high level in vascular tissues of senescent leaves (FIG. 1C).

In the present disclosure, an expression level of a gene sequence shown in SEQ ID NO: 3 was investigated, and the gene sequence shown in SEQ ID NO: 3 was overexpressed in a protoplast to obtain an amino acid sequence shown in SEQ ID NO: 1, such as to obtain results of a descriptive study of the gene sequence, which provided a basis for further improving a nitrogen uptake and assimilation efficiency.

### Example 2 Two Phenotype-Differential Allelic Haplotypes of ZmNPF6.8 in natural Populations

In 2021, 149 natural genetic populations were cultivated with a hydroponic mode in the Liuhe Experimental Greenhouse of Jiangsu Academy of Agricultural Sciences under the following conditions: light periods: 8 h/16 h (day/night), day/night temperatures: 22° C./25° C., a light intensity: about 200 μmol·m⁻²·s⁻¹, and a relative humidity: 65% to 75%. A modified Hoagland nutrient solution was adopted for cultivation, and the modified Hoagland nutrient solution had the following formula: 4.0 mM CaCl₂, 2.0 mM MgSO₄, 2.0 mM KH₂PO₄, 4.6 mM H₃BO₃, 0.5 mM MnCl₂, 0.2 mM ZnSO₄, 0.1 mM Na₆Mo₇O₂₄, 0.2 mM CuSO₄, 45 mM FeCl₃, and 5 mM KNO₃. After 2 weeks of cultivation, a total nitrogen content in leaves was determined as follows:
(1) 0.1 g of a plant leaf powder was weighed and added to a nitrogen determination tube, 1 mL of deionized water was added to the nitrogen determination tube for soaking, then 5 mL of concentrated sulfuric acid was added to the nitrogen determination tube, and the nitrogen determination tube was allowed to stand overnight. 3 independent biological replicates were set for each sample.
(2) Pyrolysis: A pyrolysis furnace was heated to 250° C., and the nitrogen determination tube was placed in the cooking furnace to allow pyrolysis for 20 min; then 10 drops of a 30% H₂O₂ catalyst were added to the nitrogen determination tube, and the nitrogen determination tube was thoroughly shaken; and the pyrolysis furnace was further heated to 350° C., and pyrolysis was further conducted for 5 h to 6 h until a transparent sample was obtained.
(3) Distillation and titration: The transparent sample was subjected to distillation in an automatic Kjeldahl nitrogen determination apparatus (Hanon) for about 5 min, a resulting product was titrated with a boric acid indicator, and a titration volume was recorded.
(4) A total nitrogen content in a plant tissue (mg/g) was calculated according to the following formula:

$$total nitrogen content = ( V - V 0 ) \times c ( HCl ) \times 14. \times {10}^{- 3} / m$$

where V represents a volume of an acid standard solution required for titration of a test sample; VO represents a volume of an acid standard solution required for titration of a blank sample; c (HCl) represents a concentration of a 0.01 mol/L standard solution, which is 0.01 mol/L; and m represents a mass of a dried sample.

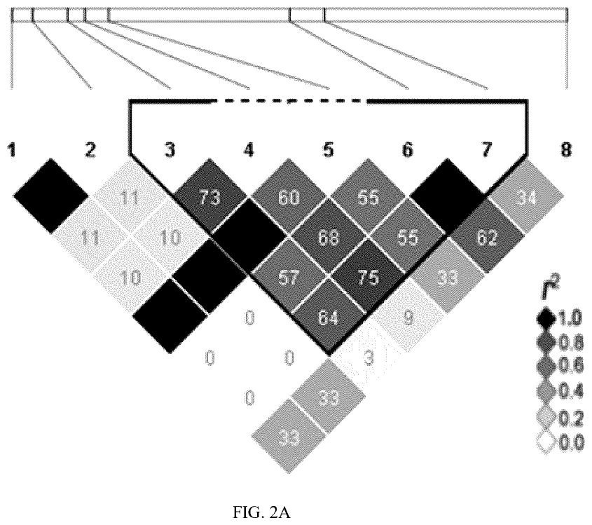

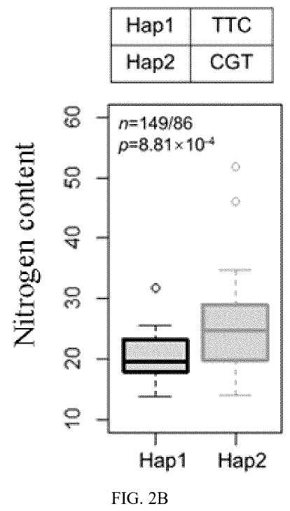

Phenotype data analysis was conducted by the SAS9.3 statistical software according to a maximum value, a minimum value, a mean value, a standard deviation, a coefficient of variation, skewness, and a kurtosis of a trait. With the TASSEL 5.0 software, total nitrogen contents and single nucleotide polymorphism (SNP) loci in natural populations of 149 Zea mays L. inbred lines were subjected to association analysis by a mixed linear model (MLM) of population structure+kinship (Q+K), where a threshold value of a significance P value was set to P<10⁻⁵. Analysis results showed that the gene ZmNPF6.8 was a major quantitative trait locus (QTL) affecting a nitrogen content in Zea mays L., and there were commonly two haplotype materials with a significant phenotypic difference: Hap1 (TTC) and Hap2 (CGT). Three SNP loci in Hap1 and Hap2 corresponded to key amino acids (Asp and Gly, Phe and Lys, and Ala and Trp). The Hap2 haplotype was a superior allelic haplotype (FIGS. 2A-2B).

### Example 3 Acquisition of a ZmNPF6.8 Transposon-Inserted Mutant Line and Determination of a Nitrogen Transport Capacity and a Yield

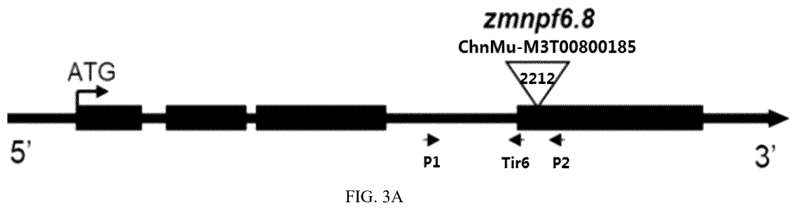

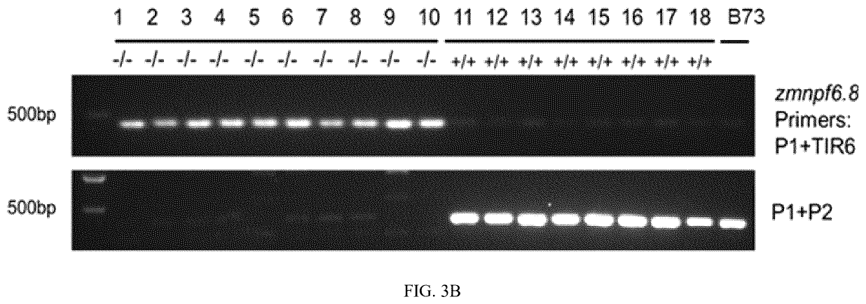

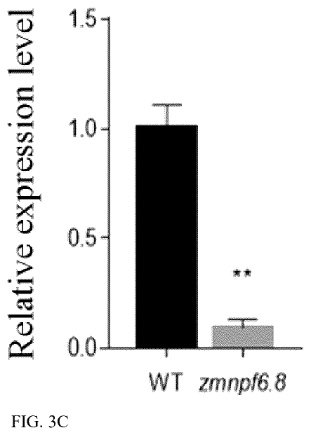

In this study, a Zea mays L. ChinaMu mutant library (http://chinamu.jaas.ac.cn/cindex.html) was constructed through hybridization of a Zea mays L. Mu active line with a B73 inbred line, and it was found through the mutant library that one Mu insertion (zmnpf6.8) occurred in an exon region 4 of the gene ZmNPF6.8 (FIG. 3A). The insertion site was at a position 181884791 of a chromosome 5. The obtained mutant library was numbered M3T00800185. In order to exclude the influence of insertion of a transposon into other sites, a mutant material was backcrossed with B73 for two generations, and a homozygous mutant (zmnpf6.8) and a WT material were acquired. A template sequence was obtained by searching Zea mays L. B73_RefGen_v4 reference genome in Phytozome (https://phytozome-next.jgi.doe.gov/).

Primer design (as shown in SEQ ID NOS: 4-6) was conducted by the primer premier 5.0 software according to the insertion site, and an annealing temperature (Tm) was about 59° C.

P1:
GAGCAGGATGGAGAGGAATAGG,
as shown in SEQ ID NO: 4;
P2:
GCGATCAACGAGACTGAGCAG,
as shown in SEQ ID NO: 5;
and
Tir6:
AGAGAAGCCAACGCCAWCGCCTCYATTTCGTC,
as shown in SEQ ID NO: 6.

The ZmNPF6.8 gene sequence was then amplified by PCR with a high-fidelity enzyme Planta® Max Super-Fidelity DNA Polymerase (Vazyme). An agarose gel with a concentration of 1.5% (M/V) was prepared as follows: agarose from Solarbio was taken, an appropriate amount of a 1× TAE buffer was added to obtain a first mixture, and the first mixture was heated in a microwave oven for 2 min to allow complete dissolution and then cooled; and 5 μ of an Evans blue (EB) dye was added to obtain a second mixture, and the second mixture was thoroughly mixed, poured into a mold, and allowed to stand for solidification. The agarose gel was transferred to an electrophoresis tank, 1× TAE was adopted as a buffer solution, and a sample was loaded; then agarose gel electrophoresis was conducted at 120 V for 1 h (FIG. 3B); and finally, ultraviolet development and photographing were conducted.

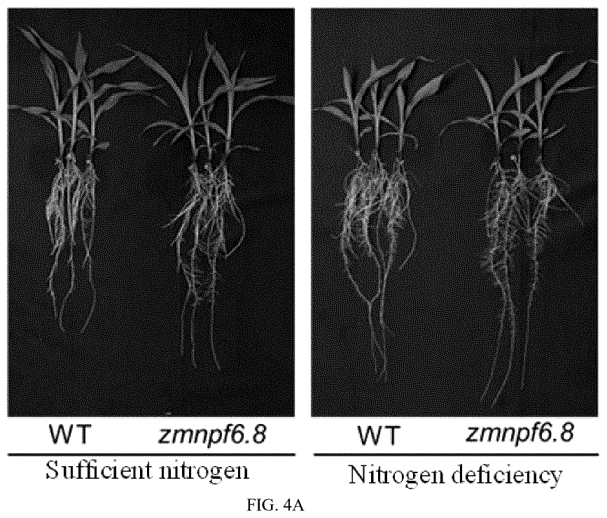

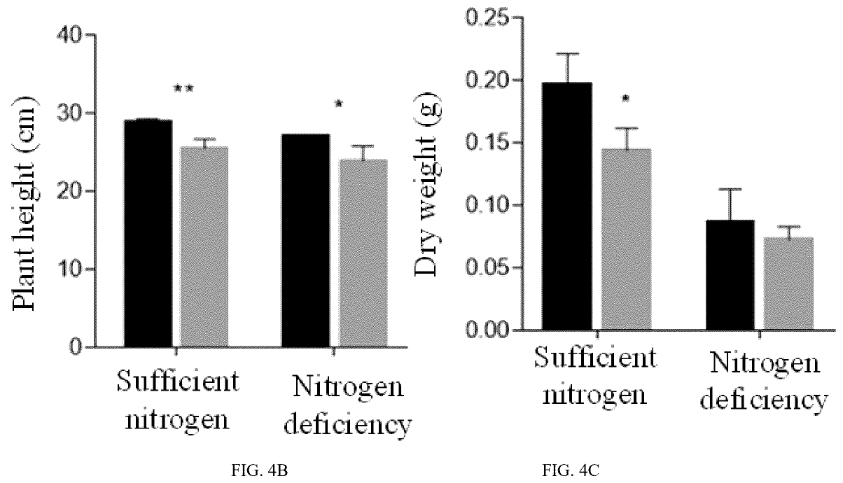

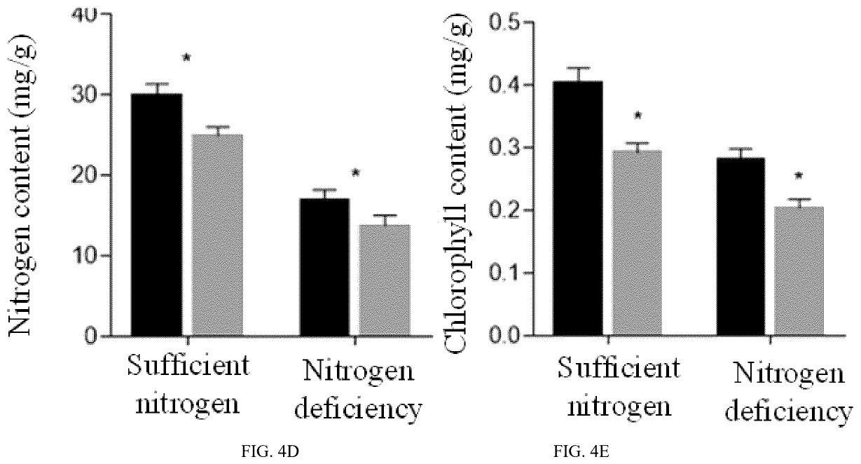

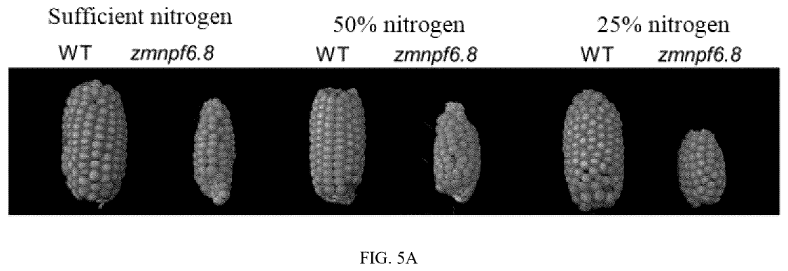

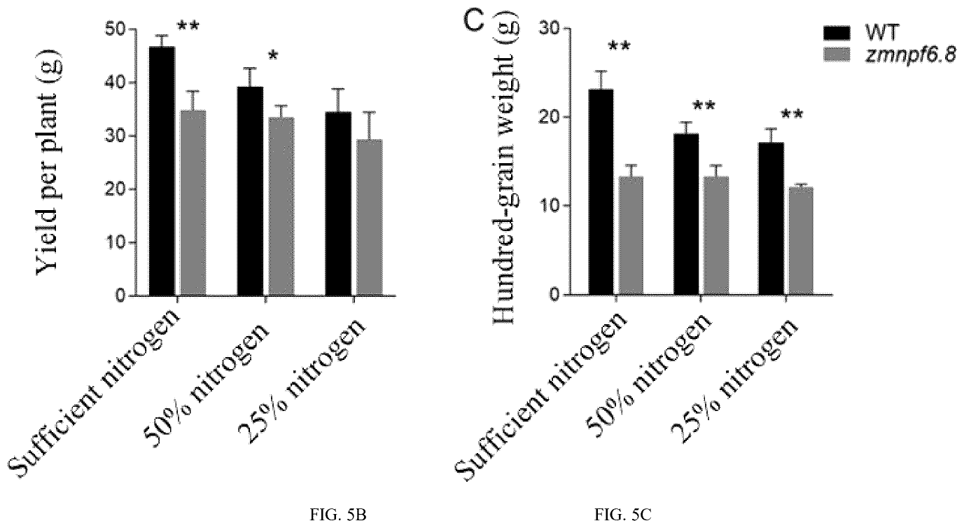

qPCR primers were designed for a mutant material with the primer premier 5.0 software (F: CGCCGTCTTCGTCGTCTTC, as shown in SEQ ID NO: 7; and R: GTTCAGCCTTCATGCCCAATTC, as shown in SEQ ID NO: 8); a CFX Connect Real-Time PCR System (Bio-Rad Laboratories) was used to conduct qRT-PCR by a two-step method; and with a housekeeping gene ZmUPF1 (Zm00001d006438) as an internal reference gene, relative expression levels of the WT material and the ZmNPF6.8 mutant material were calculated by a 2-ΔΔCt method and subjected to multi-comparison through LSD of ANOVA in the SPSS software (p <0.05). Analysis results showed that an expression level of ZmNPF6.8 in the mutant material was significantly down-regulated (FIG. 3C). Total nitrogen content-associated traits of the Zea mays L. ZmNPF6.8 mutant were investigated. In the mutant, an expression level of the gene was significantly down-regulated, and total nitrogen content and nitrate content-associated traits of the mutant decreased to varying degrees under sufficient nitrogen (SN: 5 mM KNO₃) and nitrogen deficiency (DN: 0.5 mM KNO₃) conditions (FIGS. 4A-4E). A yield of the Zea mays L. ZmNPF6.8 mutant was investigated. The yield of the mutant decreased to varying degrees under three soil fertility gradients (sufficient nitrogen, 50% nitrogen, and 25% nitrogen) (FIGS. 5A-5C).

It can be seen that the down-regulation of an expression level of the gene ZmNPF6.8 has an important impact on total nitrogen content-associated traits and a yield per plant of Zea mays L, and a nitrogen uptake and utilization efficiency of Zea mays L. and a yield per Zea mays L. plant can be regulated by changing an expression level of the gene ZmNPF6.8, which is of great significance for future breeding of a Zea mays L. variety with a high nitrogen uptake and utilization efficiency and a high yield.

Although the examples of the present disclosure have been illustrated and described, it should be understood that those of ordinary skill in the art may make various changes, modifications, replacements and variations to the above examples without departing from the principle and spirit of the present disclosure, and the scope of the present disclosure is limited by the appended claims and legal equivalents thereof.

What is claimed is:

1. A method of regulating a nitrogen uptake and utilization efficiency of Zea mays L. and a yield per Zea mays L. plant, comprising using a gene ZmNPF6.8.

2. A method of breeding a maize variety with an efficient nitrogen uptake and a high yield, comprising using a gene ZmNPF6.8.

3. The method according to claim 1, wherein the gene ZmNPF6.8 has a nucleotide sequence described in (1) or (2) below:
(1) the coding region nucleotide sequence shown in SEQ ID NO: 3; and
(2) a nucleotide sequence having 90% or more homology with the coding region nucleotide sequence shown in SEQ ID NO: 3 and encoding a same functional protein as the coding region nucleotide sequence shown in SEQ ID NO: 3.

4. The method according to claim 1, wherein the gene ZmNPF6.8 has the nucleotide sequence shown in SEQ ID NO: 2.

5. The method according to claim 1, wherein a protein encoded by the gene ZmNPF6.8 has an amino acid sequence described in (1) or (2) below:
(1) the amino acid sequence shown in SEQ ID NO: 1; and
(2) an amino acid sequence obtained through substitution and/or deletion and/or addition of one or more amino acid residues based on the amino acid sequence shown in SEQ ID NO: 1 and having 90% or more homology with and a same function as the amino acid sequence shown in SEQ ID NO: 1.

6. The method according to claim 5, wherein an expression level of the protein encoded by the gene ZmNPF6.8 is changed to regulate the nitrogen uptake and utilization efficiency of the Zea mays L. and the yield per Zea mays L. plant; and the expression level of the protein encoded by the gene ZmNPF6.8 is changed by: (1) artificially adding a strong promoter element Ubiquitin or CaMV35S; (2) changing an expression of the gene ZmNPF6.8 through mutagenesis of a transposon, a fast neutron, and ethyl methane sulfonate (EMS); and (3) changing the expression of the gene ZmNPF6.8 through interference, silencing, inhibition, targeted knockout, or site-directed mutagenesis.

7. The method according to claim 6, wherein a Mutator transposon insertion method is adopted to change expression levels of mRNA with a normal activity of the gene ZmNPF6.8 and the protein encoded by the gene ZmNPF6.8.

8. The method according to claim 2, wherein the gene ZmNPF6.8 has a nucleotide sequence described in (1) or (2) below:
(1) the coding region nucleotide sequence shown in SEQ ID NO: 3; and
(2) a nucleotide sequence having 90% or more homology with the coding region nucleotide sequence shown in SEQ ID NO: 3 and encoding a same functional protein as the coding region nucleotide sequence shown in SEQ ID NO: 3.

9. The method according to claim 2, wherein the gene ZmNPF6.8 has the nucleotide sequence shown in SEQ ID NO: 2.

10. The method according to claim 2, wherein a protein encoded by the gene ZmNPF6.8 has an amino acid sequence described in (1) or (2) below:
(1) the amino acid sequence shown in SEQ ID NO: 1; and
(2) an amino acid sequence obtained through substitution and/or deletion and/or addition of one or more amino acid residues based on the amino acid sequence shown in SEQ ID NO: 1 and having 90% or more homology with and a same function as the amino acid sequence shown in SEQ ID NO: 1.


SEQUENCE LISTING (8 sequences)

SEQ ID NO: 1 - protein, 596 aa, synthetic construct
  REGION 1..596: note = The sequence is synthesized.
MVSGAGHGGYGGGDDGQAVDFRGNPADKSRTGGWLGAGLILGTELAERVCVMGISMNLVTYLVGELHLSNSKSATVVTNFMGTLNLLALVGGFLADAKLGRYLTIAISATIAATGVSLLTVDTTVPSMRPPACLDARGPRAHECVPARGGQLALLYAALYTVAAGAGGLKANVSGFGSDQFDGRDPREERAMVFFFNRFYFCVSLGSLFAVTVLVYVQDNVGRGWGYGVSAVAMALAVAVLVAGTPRYRYRRPQGSPLTAVGRVLAAAWRKRRLPLPADAAELHGFAAAKVAHTDRLRWLDKAAIVEAEPAGKQRASAAAASTVTEVEEVKMVAKLLPIWSTCILFWTVYSQMTTFSVEQATRMDRHLRPGSGAGGFAVPAGSFSVFLFLSILLFTSLNERLLVPLAARLTGRPQGLTSLQRVGAGLALSVAAMAVSALVERKRRDAANGPGHVAVSAFWLVPQYFLVGAGEAFAYVGQLEFFIREAPERMKSMSTGLFLVTLSMGFFLSSLLVFAVDAATAGTWIRNNLDRGRLDLFYWMLALLGVANFAVFVVFARRHQYKATSLPASVAPDGTGHKEMDDFVAVTEAVEGVDV

SEQ ID NO: 2 - DNA, 3227 bp, synthetic construct
  misc_feature 1..3227: note = The sequence is synthesized.
  source 1..3227: mol_type = other DNA
ttccgagtagcagtgcaggagcagccacagccacgccctcctatctagctataaatcctcacccggcctcatccctcttggacaaactcacagcacaaacgttcctctcctcttccatcgtcgtcctcgccgctcccggccccgcctgtctagtcccctcttctcgcgtgcaacccctgtactgtacgtgctaggcacacagctagctgcagctacagctgcagggcagcaaatggtaagagccgcacaacgcacatcctcgctctctgtctaatgcatggtggagatagctctgatggcgtatatacgtacctgaacttctgaacacgtacgtgtgtaggtttccggtgcgggtcatggtgggtacggcggcggcgacgacgggcaggccgtggacttccggggcaacccggcggacaagtcgaggaccggaggctggctgggcgccgggctgatcctgggcacggagctggcggagcgcgtgtgcgtgatgggcatctcgatgaacctggtgacgtacctcgtcggcgagctgcacctctccaactccaagtccgccaccgtggtgaccaacttcatgggcacgctcaacctgctcgccctcgtcggcggcttcctcgccgacgccaagctcggccgctacctcaccatcgccatctccgccacaatcgccgccacggtatacatcatgttggtgtcgtgtcgtcgcattgcgcgactaattctaatctaatctagcggaacggtgacatggcacagggcgtgagcttgctgacggtggacacgacggtgccgagcatgcgtccgccggcgtgcctggacgcccgcgggccgcgcgcgcacgagtgcgtgccggcgcgcggcgggcagctggcgctgctgtacgcggcgctgtacacggtggcggcgggggccggcgggctcaaggcgaacgtgtccgggttcgggtcggaccagttcgacgggcgcgacccgcgggaggagcgcgccatggtgttcttcttcaaccgcttctacttctgcgtcagcctggggtcgctgttcgcggtcaccgtgctggtgtacgtgcaggacaacgtggggcggggctggggctacggcgtctccgcagtcgccatggcgctcgccgtcgccgtgctcgtggccggcacgccccggtacaggtaccgccgcccgcagggcagcccgctgacggcggtcggccgggtgctcgccgcggcgtggaggaagcgccggctgccgctgcccgccgacgccgccgagctccacgggttcgccgcggccaaggtcgcacacactgacaggctcaggtaacgcgacgcgtgctctccttttgatttttgcctgccgtcgcgttgcgttcctttgatcacgtctctgtcttagcagctagcagcagccagcgaccaattaatctacggtgctcatctcatctagtagtatagtattagcttagctcgaggcaaataaacagacaccataataaattaaagggttagggttgggatgcatatagtactggggcacacgaagttttatgtcctgcaggggacacgatgacgtcctagcagtacgaccgatcgagctagtaaccgctgctgcagagatcacgcgcgagttgggcaggtctattgtaagtacgatcacatgccacttgtccgagcctcgtgtcagtgtcacggctgaaatgcagagaagccgagaggggtaccccttgacgccggtcgctaacacaacacgcaaaagagggaattattgaacccagtcgctaacacaacggaacttgaaagaaaaaaaaggatctagacaggtagacgtgccgcgaaagctcgttacactgttccgatccggccggaaaactggccgatgtgccggcctttcagctgtcgctctcgctgctgagtcacccagtggtgacactgacagcacgggcgcgtgatgcgtcacgcgatcaacgagactgagcagtagtgcggtactgtagtctgtagctgtagtagagtacgtgtacgtgtgtgcctctcgcgcgcgctacagttgacgaatgcatcatcatcatgcaggtggcttgacaaggcggcgatcgtggaggccgagccggcggggaagcagcgggcgagcgcggcggcggcgtcgacggtgacggaggtggaggaggtgaagatggtggcgaagctgctgcccatctggtccacgtgcatcctcttctggacggtctactcccagatgaccaccttctcggtggagcaggccacgcgcatggaccgccacctgcgcccgggctccggcgccggcggcttcgccgtcccggcgggctccttctccgtcttcctattcctctccatcctgctcttcacctcgctcaacgagcgcctcctcgtgccgctggccgcccgcctcacgggccgcccgcaggggctcacctcgctgcagcgcgtcggggccgggctcgcgctctccgtcgccgccatggccgtctccgcgctcgtcgagaggaagcggcgcgacgcggccaacgggccgggccacgtcgccgtcagcgccttctggctcgtcccgcagtacttcctcgtcggcgccggcgaggccttcgcctacgtgggccagctggagttcttcatccgcgaggcgcccgagcggatgaagtccatgagcaccggcctcttcctcgtcacgctctccatgggcttcttcctcagcagcttgctcgtcttcgccgtcgacgccgccaccgcgggcacgtggatccggaacaacctcgaccgcggcaggctcgacctcttctactggatgctggccctgctcggcgtcgccaacttcgccgtcttcgtcgtcttcgccaggcggcaccagtacaaggccaccagcttgccggcgtcggtggcgcccgacggcaccgggcacaaggagatggacgacttcgtcgcagtcacggaggccgtggaaggagtggacgtgtagggcgcgcatgtcaggctcacacttcatcggtgggtggcctctgctgtttaattttgcctttgggtagttagatcaacaagatattgaattgggcatgaaggctgaacaccatgtgtgtgcattacagtacccaacccaaactctaacatagcatcgttataacttataatggctgcgttgtaatgtggcgttaggccatgttcactctaatactacctcagtcccgaaatataattcattttagtcttttaatgaattcatacaat

SEQ ID NO: 3 - DNA, 1791 bp, synthetic construct
  misc_feature 1..1791: note = The sequence is synthesized.
  source 1..1791: mol_type = other DNA
atggtttccggtgcgggtcatggtgggtacggcggcggcgacgacgggcaggccgtggacttccggggcaacccggcggacaagtcgaggaccggaggctggctgggcgccgggctgatcctgggcacggagctggcggagcgcgtgtgcgtgatgggcatctcgatgaacctggtgacgtacctcgtcggcgagctgcacctctccaactccaagtccgccaccgtggtgaccaacttcatgggcacgctcaacctgctcgccctcgtcggcggcttcctcgccgacgccaagctcggccgctacctcaccatcgccatctccgccacaatcgccgccacgggcgtgagcttgctgacggtggacacgacggtgccgagcatgcgtccgccggcgtgcctggacgcccgcgggccgcgcgcgcacgagtgcgtgccggcgcgcggcgggcagctggcgctgctgtacgcggcgctgtacacggtggcggcgggggccggcgggctcaaggcgaacgtgtccgggttcgggtcggaccagttcgacgggcgcgacccgcgggaggagcgcgccatggtgttcttcttcaaccgcttctacttctgcgtcagcctggggtcgctgttcgcggtcaccgtgctggtgtacgtgcaggacaacgtggggcggggctggggctacggcgtctccgcagtcgccatggcgctcgccgtcgccgtgctcgtggccggcacgccccggtacaggtaccgccgcccgcagggcagcccgctgacggcggtcggccgggtgctcgccgcggcgtggaggaagcgccggctgccgctgcccgccgacgccgccgagctccacgggttcgccgcggccaaggtcgcacacactgacaggctcaggtggcttgacaaggcggcgatcgtggaggccgagccggcggggaagcagcgggcgagcgcggcggcggcgtcgacggtgacggaggtggaggaggtgaagatggtggcgaagctgctgcccatctggtccacgtgcatcctcttctggacggtctactcccagatgaccaccttctcggtggagcaggccacgcgcatggaccgccacctgcgcccgggctccggcgccggcggcttcgccgtcccggcgggctccttctccgtcttcctattcctctccatcctgctcttcacctcgctcaacgagcgcctcctcgtgccgctggccgcccgcctcacgggccgcccgcaggggctcacctcgctgcagcgcgtcggggccgggctcgcgctctccgtcgccgccatggccgtctccgcgctcgtcgagaggaagcggcgcgacgcggccaacgggccgggccacgtcgccgtcagcgccttctggctcgtcccgcagtacttcctcgtcggcgccggcgaggccttcgcctacgtgggccagctggagttcttcatccgcgaggcgcccgagcggatgaagtccatgagcaccggcctcttcctcgtcacgctctccatgggcttcttcctcagcagcttgctcgtcttcgccgtcgacgccgccaccgcgggcacgtggatccggaacaacctcgaccgcggcaggctcgacctcttctactggatgctggccctgctcggcgtcgccaacttcgccgtcttcgtcgtcttcgccaggcggcaccagtacaaggccaccagcttgccggcgtcggtggcgcccgacggcaccgggcacaaggagatggacgacttcgtcgcagtcacggaggccgtggaaggagtggacgtgtag

SEQ ID NO: 4 - DNA, 22 bp, synthetic construct
  misc_feature 1..22: note = The sequence is synthesized.
  source 1..22: mol_type = other DNA
gagcaggatggagaggaatagg

SEQ ID NO: 5 - DNA, 21 bp, synthetic construct
  misc_feature 1..21: note = The sequence is synthesized.
  source 1..21: mol_type = other DNA
gcgatcaacgagactgagcag

SEQ ID NO: 6 - DNA, 32 bp, synthetic construct
  misc_feature 1..32: note = The sequence is synthesized.
  source 1..32: mol_type = other DNA
agagaagccaacgccawcgcctcyatttcgtc

SEQ ID NO: 7 - DNA, 19 bp, synthetic construct
  misc_feature 1..19: note = The sequence is synthesized.
  source 1..19: mol_type = other DNA
cgccgtcttcgtcgtcttc

SEQ ID NO: 8 - DNA, 22 bp, synthetic construct
  misc_feature 1..22: note = The sequence is synthesized.
  source 1..22: mol_type = other DNA
gttcagccttcatgcccaattc